(19) United States
(12) Patent Application Publication
(10) Pub. No.: US 2025/0339603 A1
(43) Pub. Date: Nov. 6, 2025

(54) MODULAR NEGATIVE PRESSURE WOUND THERAPY DEVICES, SYSTEMS, AND METHODS

(71) Applicant: Solventum Intellectual Properties Company, Maplewood, MN (US)

(72) Inventors: Benjamin A. PRATT, Bracknell (GB); Robert HOWARD, Bracknell (GB); James K. SEDDON, Bracknell (GB); Richard M. KAZALA, San Antonio, TX (US); Michael R. OLIVA, Saint Paul, MN (US); Larry T. RANDOLPH, Saint Paul, MN (US)

(21) Appl. No.: 18/867,082

(22) PCT Filed: May 4, 2023
(86) PCT No.: PCT/IB2023/054646

§ 371 Date: Nov. 19, 2024

Related U.S. Application Data
(60) Provisional application No. 63/345,262, filed on May 24, 2022.

(51) Int. Cl. A61M 1/00

(52) U.S. Cl. CPC A61M 1/966; A61M 1/912; A61M 1/98; A61M 2205/8206

(57) ABSTRACT

Apparatuses, systems, and methods in a negative pressure wound therapy environment. A therapy device can include a pump housing and a power module configured to couple to the pump housing. The pump housing comprises a pump configured to deliver negative pressure to a dressing, a first pressure sensor configured to sense a pressure at the pump, a controller operably coupled to the pump and the first pressure sensor within an interior of the pump housing, and a first communications connector configured to provide communication with the controller from an exterior of the pump housing. The power module comprises a battery configured to provide power, a second pressure sensor configured to sense an ambient pressure, and a second communications connector configured to be coupled to the first communications connector to operably connect the battery and the second pressure sensor to the controller.

### CROSS-REFERENCE TO RELATED APPLICATIONS

This application is a U.S. National Stage Entry of International Application No. PCT/IB2023/054646, filed on May 4, 2023, which claims the benefit of priority to U.S. Provisional Application No. 63/345,262, filed on May 24, 2022, each of which are incorporated herein by reference in their entirety.

### TECHNICAL FIELD

The invention set forth in the appended claims relates generally to tissue treatment systems and more particularly, but without limitation, to modular negative pressure wound therapy devices, systems and methods.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for modular therapy devices in a negative-pressure therapy environment are set forth in the appended claims. Illustrative embodiments are also provided to enable a person skilled in the art to make and use the claimed subject matter.

For example, in some embodiments, a therapy device is described. The therapy device can include a pump housing and a power module configured to couple to the pump housing. The pump housing can include a pump configured to deliver negative pressure to a dressing, a first pressure sensor configured to sense a pressure at the pump, a controller operably coupled to the pump and the first pressure sensor within an interior of the pump housing, and a first communications connector configured to provide communication with the controller from an exterior of the pump housing. The power module can include a battery configured to provide power, a second pressure sensor configured to sense an ambient pressure, and a second communications connector configured to be coupled to the first communications connector to operably connect the battery and the second pressure sensor to the controller.

In some example embodiments, the pump housing can include a user interface.

In some example embodiments, the power module can include a user interface.

In some example embodiments, the pump housing can include a wireless communication device operably coupled to the controller. The wireless communication device can be a Bluetooth® device.

In some example embodiments, the pump housing can further include a dressing connection configured to fluidly couple the pump to the dressing.

In some example embodiments, the therapy device can further include a pressure sensing module. The pressure sensing module can be configured to be coupled between the pump housing and the power module. In some example embodiments, the therapy device can further include a fluid storage container configured to be fluidly coupled between the pump and the dressing. In some example embodiments, the pressure sensing module can include a third pressure sensor and a third communications connector. The third communications connector can be configured to be coupled between the first communications connector and the second communications connector to operably connect the third pressure sensor to the controller. In some example embodiments, the therapy device can further include a dressing connector configured to couple the therapy device to the dressing. The dressing connector can include a first connector, a second connector, a negative pressure pathway, and a sensing pathway. The first connector can be configured to couple to the pump housing in fluid communication with the pump. The second connector can be configured to couple to the pressure sensing module in fluid communication with the third pressure sensor. The negative pressure pathway can be configured to fluidly couple the first connector and the pump to the dressing. The sensing pathway can be configured to fluidly couple the second connector and the third pressure sensor to the dressing. In some example embodiments, at least a portion of the negative pressure pathway and the sensing pathway are carried by a multi-lumen conduit. In some example embodiments, the sensing pathway can be fluidly isolated from the negative pressure pathway. In some example embodiments, the therapy device can further include a fluid storage container configured to be coupled in fluid communication with the negative pressure pathway between the pump and the dressing. In some example embodiments, the therapy device can further include a second pump in fluid communication with the negative pressure pathway. The second pump can be positioned within the pressure sensing module.

In some example embodiments, the therapy device can further include a blockage detection module. The blockage detection module can be configured to detect blockages within the dressing or between the therapy device and the dressing.

In some example embodiments, the pump housing can have an interior chamber that can be hermetically sealed from ambient environment.

Also described herein is a system for treating a tissue site with negative pressure. In some example embodiments, the system can include a dressing, a pump housing, and a power module configured to couple to the pump housing. In some example embodiments, the pump housing can include a pump configured to deliver negative pressure to the dressing and a first communications connector configured to provide communication with the pump from an exterior of the pump housing. In some example embodiments, the power module can include a battery configured to provide power, a first pressure sensor configured to sense a pressure at the pump, a second pressure sensor configured to sense an ambient pressure, a controller operably coupled to the battery, the first pressure sensor, and the second pressure sensor within an interior of the power module, and a second communications connector configured to be coupled to the first communications connection to operably connect the controller and the first pressure sensor to the pump.

Also described herein is a method of treating a tissue site with negative pressure. In some example embodiments, the method can include obtaining a pump housing that can include a pump configured to generate negative pressure, a first pressure sensor configured to sense a pressure at the pump, and a controller operably coupled to the pump and the first pressure sensor within an interior of the pump housing. The method can further include obtaining a power module including a battery configured to provide power and a second pressure sensor configured to sense an ambient pressure. The method can further include coupling the pump housing to the power module to form a therapy device. The battery and the second pressure sensor can be operably connected to the controller. The method can further include fluidly coupling the pump to a dressing disposed at the tissue site, actuating the pump to deliver negative pressure to the dressing, and monitoring, with the therapy device, the dressing while the pump is delivering negative pressure to the dressing.

In some example embodiments, the method can further include coupling a pressure sensing module between the pump housing and the power module of the therapy device. The pressure sensing module can include a third pressure sensor. In some example embodiments, coupling the therapy device to the dressing disposed at the tissue site can include coupling the therapy device to a dressing connector. The dressing connector can include a first connector, a second connector, a negative pressure pathway, and a sensing pathway. The first connector can be configured to couple to the pump housing in fluid communication with the pump. The second connector can be configured to couple to the pressure sensing module in fluid communication with the third pressure sensor. The negative pressure pathway can be configured to fluidly couple the first connector and the pump to the dressing. The sensing pathway can be configured to fluidly couple the second connector and the third pressure sensor to the dressing. In some example embodiments, the method can further include collecting fluid from the tissue site in a canister of the therapy device. The canister can be configured to be coupled in fluid communication with the negative pressure pathway between the pump and the dressing. In some example embodiments, monitoring with the therapy device, the dressing while the pump is delivering negative pressure to the dressing can include monitoring the pressure at the tissue site with the pressure sensing module.

In some example embodiments, monitoring, with the therapy device, the dressing while the pump is delivering negative pressure to the dressing can include using a blockage detection module of the therapy device to detect blockages within the dressing or between the therapy device and the dressing.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2A is a front view of a therapy device that may be associated with some embodiments of the therapy system of FIG. 1;

FIG. 2B is a top view of the therapy device of FIG. 2A;

FIG. 2C is a side view of the therapy device of FIG. 2A;

FIG. 2D is a front exploded view of the therapy device of FIG. 2A;

FIG. 2E is a perspective exploded view of the therapy device of FIG. 2A;

FIG. 2F is a cut-away front view of the therapy device of FIG. 2A;

FIG. 3A is a front view of another example embodiment of a therapy device that may be associated with some embodiments of the therapy system of FIG. 1;

FIG. 3B is a side view of the therapy device of FIG. 3A;

FIG. 4 is a perspective view of an example embodiment of the therapy system of FIG. 1;

FIG. 5 is a perspective partial cut-away view of an example embodiment of the therapy device of FIG. 2A;

FIG. 6 is a front cut-away view of another example embodiment of a therapy device of FIG. 1;

FIG. 7A is a front exploded view of another example embodiment of a therapy device including a pressure sensing module that may be associated with some embodiments of the therapy system of FIG. 1;

FIG. 7B is a side cut-away view of the therapy device of FIG. 7A;

FIG. 7C is a front cut-away view of the therapy device of FIG. 7A;

FIG. 7D is a perspective exploded view of the pressure sensing module of FIG. 7A;

FIG. 7E is a back view of the therapy device of FIG. 7A;

FIG. 8A is a front view of the therapy device of FIG. 7A coupled to a dressing connector;

FIG. 8B is an exploded view of the therapy device and the dressing connector of FIG. 8A;

FIG. 8C is a side view of the therapy device and the dressing connector of FIG. 8A;

FIG. 9 is another example embodiment of the therapy system of FIG. 1;

FIG. 10A is another example embodiment of the therapy system of FIG. 1; and

FIG. 10B is a side view of the therapy device of the therapy system of FIG. 10A.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

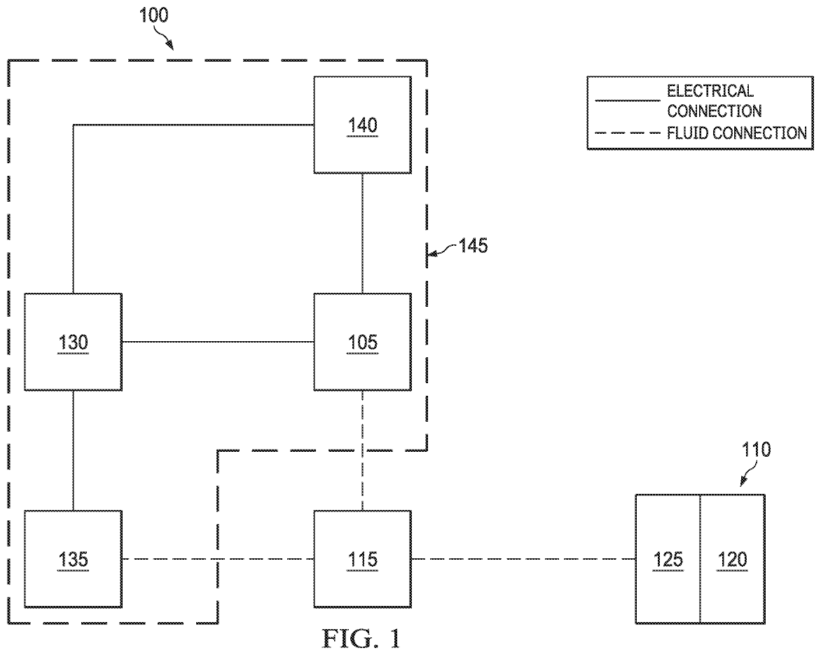

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit or a therapy device 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inspire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example.

In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

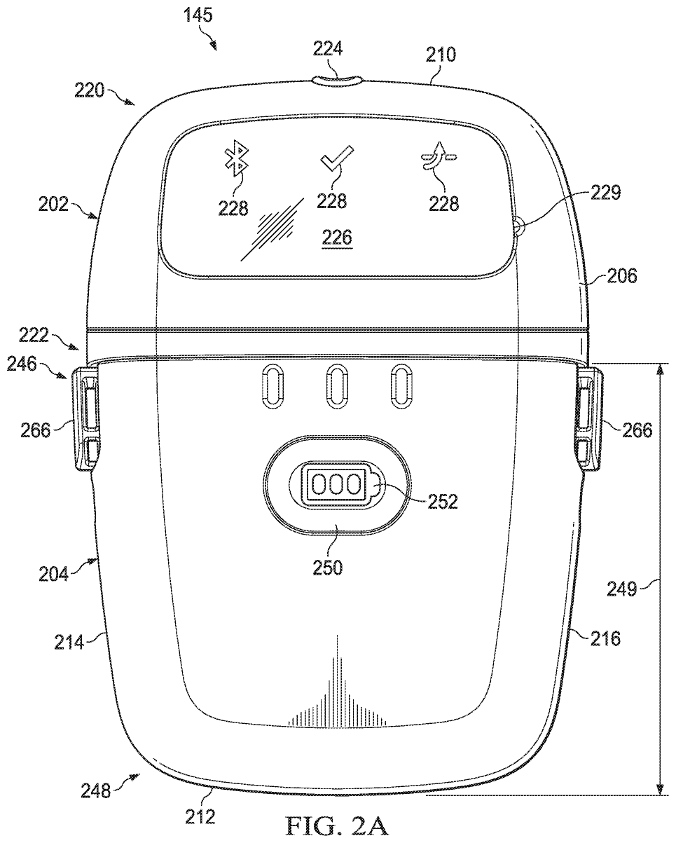

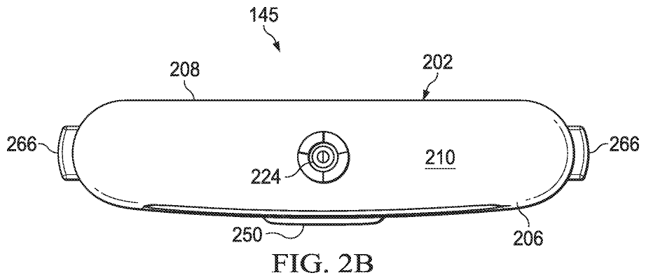

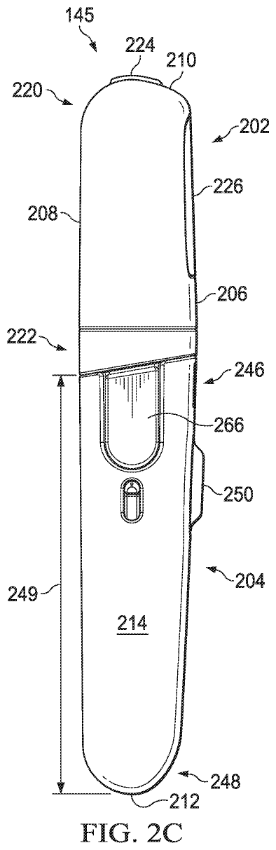

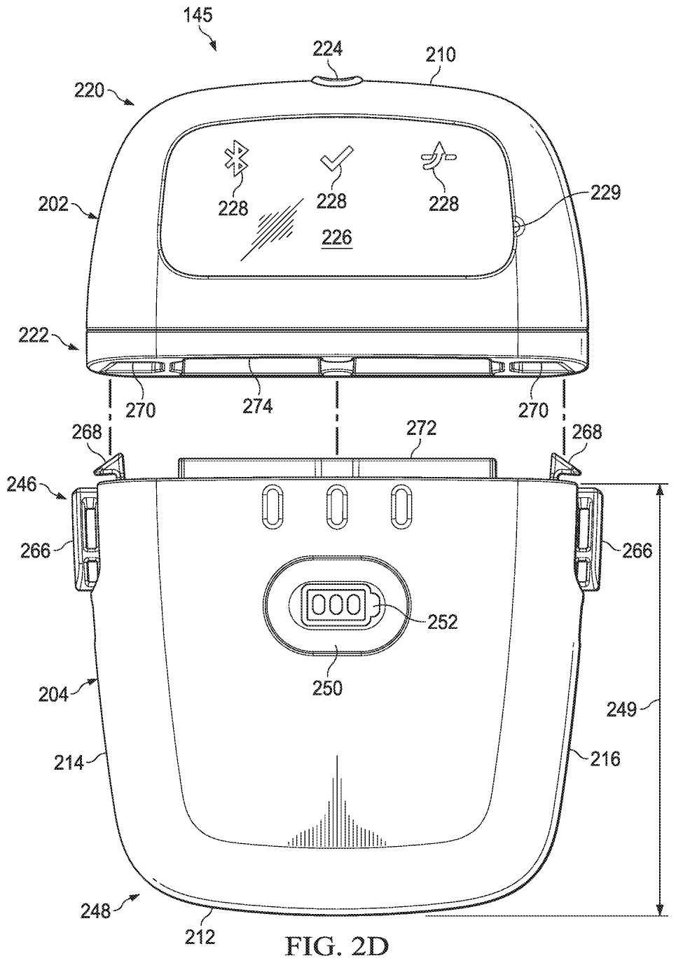

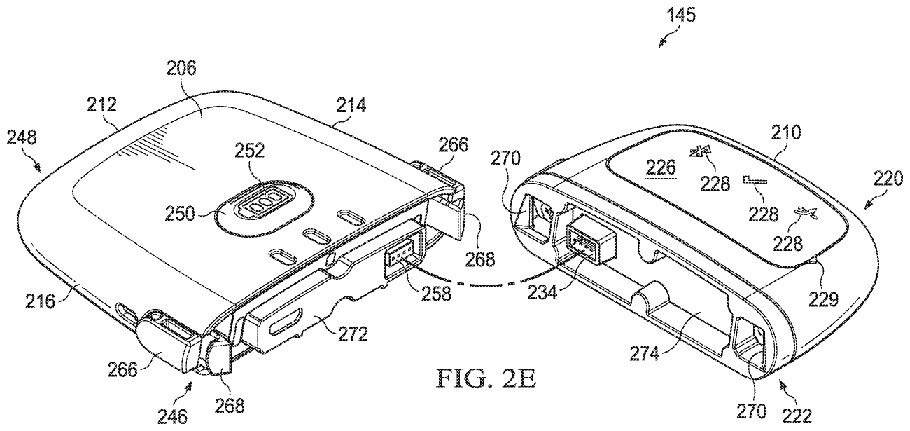

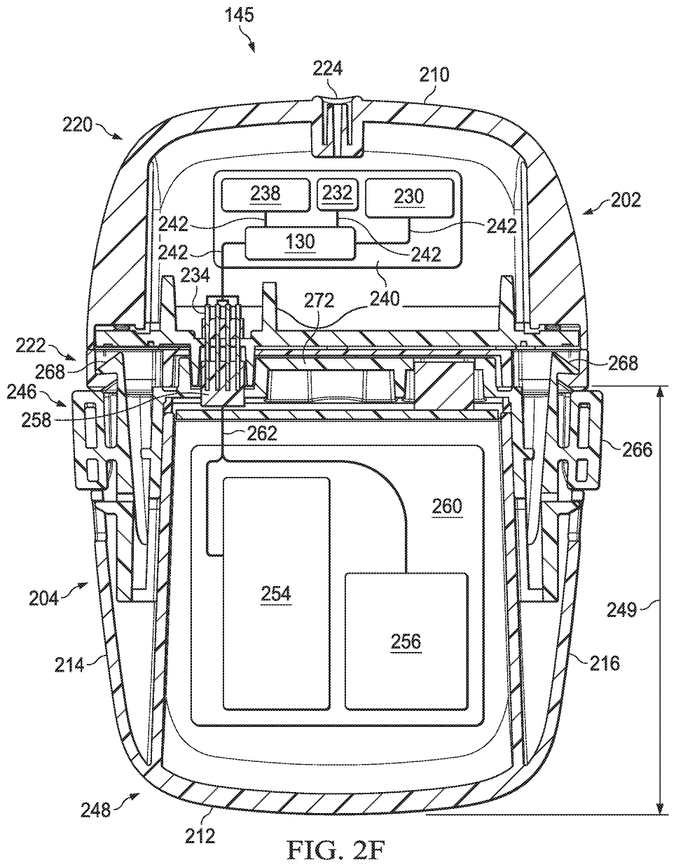

Referring collectively to FIGS. 2A-2F, these figures illustrate multiple views of an example embodiment of the therapy device 145. FIG. 2A is a front view of the therapy device 145. FIG. 2B is a top view of the therapy device 145. FIG. 2C is a side view of the therapy device 145. FIG. 2D is an exploded front view of the therapy device 145. FIG. 2E is an exploded perspective view of the therapy device 145. FIG. 2F is a front cut-away view of the therapy device 145.

The therapy device 145 may include a pump housing 202 and a power module 204. In some embodiments, the therapy device 145 may be substantially rectangular. In other embodiments, the therapy device 145 may be ovular, square, or another shape. The therapy device 145 may have a first surface 206, a second surface 208 opposite the first surface 206, a top surface 210 that may couple the first surface 206 and the second surface 208, a bottom surface 212 opposite the top surface 210, a first side 214 that may couple the top surface 210 and the bottom surface 212 and the first surface 206 and the second surface 208, and a second side 216 opposite the first side 214.

The pump housing 202 may have a first end 220 and a second end 222 opposite the first end 220. The first end 220 of the pump housing 202 may include the top surface 210 of the therapy device 145. There may be a connection point or a dressing connection 224 on the first end 220 of the pump housing 202 in some embodiments. The dressing connection 224 may couple the pump housing 202 to the dressing 110 in some embodiments. For example, a conduit may be coupled between the dressing connection 224 and the dressing 110.

The pump housing 202 may optionally include a user interface 226 in some embodiments. The user interface 226 may be configured to output information related to the therapy device 145. In some embodiments, a user may be able to interact with or control the therapy device 145 via the user interface 226. For example, the user interface 226 may include a touch screen or buttons that allow a user to communicate with the therapy device 145. In some embodiments, the user interface 226 may further include one or more indicators 228. The indicators 228 may be symbols that may change color or appearance when a certain condition is met. For example, one of the indicators 228 may be a Bluetooth® symbol that may light up when the therapy device 145 is communicating with an exterior device via Bluetooth®. In some embodiments, one of the indicators 228 may be a check mark that may light up when the pump housing 202 is coupled to the power module 204. The check mark may instead light up when the therapy device 145 is powered on. In some embodiments, one of the indicators 228 may be an airflow symbol that may indicate when a pump is actuated within the therapy device 145. In some embodiments, the one or more indicators 228 may be different symbols that may communicate information about the therapy device 145 to a user.

In some embodiments, the pump housing 202 may further include a port 229. The port 229 may provide communication between an ambient environment and the pump housing 202. In some embodiments the port 229 may be disposed on the first surface 206 of the pump housing 202. In other embodiments, the port 229 may be at a different location of the pump housing 202.

In some embodiments, the pump housing 202 may include the controller 130, a pump 230, a first pressure sensor 232, and a first communications connector 234. In some embodiments, the pump 230 may be the negative-pressure source 105 and may be configured to deliver a negative pressure to the dressing 110 when the pump 230 is actuated. The first pressure sensor 232 may be configured to sense a pressure at the pump 230. The first communications connector 234 may be configured to provide communication with the controller 130 from an exterior of the pump housing 202. For example, the first communications connector 234 may be configured to communicatively couple with the power module 204 of the therapy device 145. In some embodiments, the pump housing 202 may further include a wireless communication device 238. The wireless communication device 238 may be a Bluetooth® device in some embodiments. The wireless communication device 238 may be configured to send and receive signals from an exterior of the pump housing 202.

The controller 130, the pump 230, the first pressure sensor 232, and the wireless communication device 238 may each be coupled to a printed circuit board 240. Further, the pump 230, the first pressure sensor 232, and the wireless communication device 238 may each be communicatively coupled to the controller 130. Further, the controller 130 may be communicatively coupled with the first communications connector 234. In some embodiments, there may be wires 242 coupling the pump 230, the first pressure sensor 232, and the wireless communication device 238 to the controller 130. In other embodiments, the components may be wirelessly coupled. The controller 130 may be configured to send signals to and receive signals from each of the pump 230, the first pressure sensor 232, and the wireless communication device 238.

The power module 204 may have a first end 246 and a second end 248 opposite the first end 246. The power module 204 may have a length 249 defined between the first end 246 of the power module 204 and the second end 248 of the power module 204. The second end 248 of the power module 204 may include the bottom surface 212 of the therapy device 145. The power module 204 may optionally include a user interface 250 in some embodiments. The user interface 250 may be configured to output information related to the therapy device 145. In some embodiments, a user may be able to interact with or control the therapy device 145 via the user interface 250. For example, the user interface 250 may include a touch screen or buttons that allow a user to communicate with the therapy device 145. In some embodiments, the user interface 250 may be or may include a battery indicator 252. The battery indicator 252 may output a battery level to a user.

The power module 204 may include a battery 254, a second pressure sensor 256, and a second communications connector 258. The battery 254 may be configured to provide power to the therapy device 145. The battery 254 may be rechargeable and/or may be replaceable. The second pressure sensor 256 may be configured to sense an ambient pressure. In some embodiments, the battery 254 and the second pressure sensor 256 may be coupled to a printed circuit board 260. The battery 254 and the second pressure sensor 256 may be communicatively coupled to the second communications connector 258. In some embodiments, there may be wires 262 coupling the battery 254 and the second pressure sensor 256 to the second communications connector 258. In other embodiments, the components may be wirelessly coupled. The second communications connector 258 may be coupled with the first communications connector 234. When the second communications connector 258 is coupled to the first communications connector 234, the battery 254 and the second pressure sensor 256 may be communicatively coupled to the controller 130. In some embodiments, when the second communications connector 258 is coupled to the first communications connector 234 the controller 130 may be configured to send signals to and receive signals from the battery 254 and the second pressure sensor 256.

In some embodiments, the second end 222 of the pump housing 202 may be coupled to the first end 246 of the power module 204 with snaps 266. In some embodiments, the therapy device 145 may include one of the snaps 266 on the first side 214 of the therapy device 145 and another of the snaps 266 on the second side 216 of the therapy device 145. The snaps 266 may enable a user to release the pump housing 202 from the power module 204. In some embodiments, the snaps 266 may be part of the power module 204 and may be depressed into the power module 204 to release the pump housing 202 from the power module 204. When the snaps 266 are depressed into the power module 204, hooks 268 may be released from recesses 270 of the pump housing 202. In other embodiments, the snaps 266 may be manipulated in another way to release the power module 204 from the pump housing 202. In other embodiments, not pictured herein, the snaps 266 may be disposed within the pump housing 202 or the power module 204 such that a user cannot access the snaps 266. When disposed within the pump housing 202 or the power module 204 the snaps 266 may couple the pump housing 202 to the power module 204 but the pump housing 202 and the power module 204 may not be separated by a user.

In some embodiments, the power module 204 may further include an extension 272 that may extend into a center recess 274 of the pump housing 202 when the pump housing 202 and the power module 204 are coupled together. In some embodiments, the second communications connector 258 may extend into the extension 272 and the first communications connector 234 may extend into the center recess 274. When the pump housing 202 and the power module 204 are coupled, the first communications connector 234 and the second communications connector 258 may couple together to provide communication between the pump housing 202 and the power module 204.

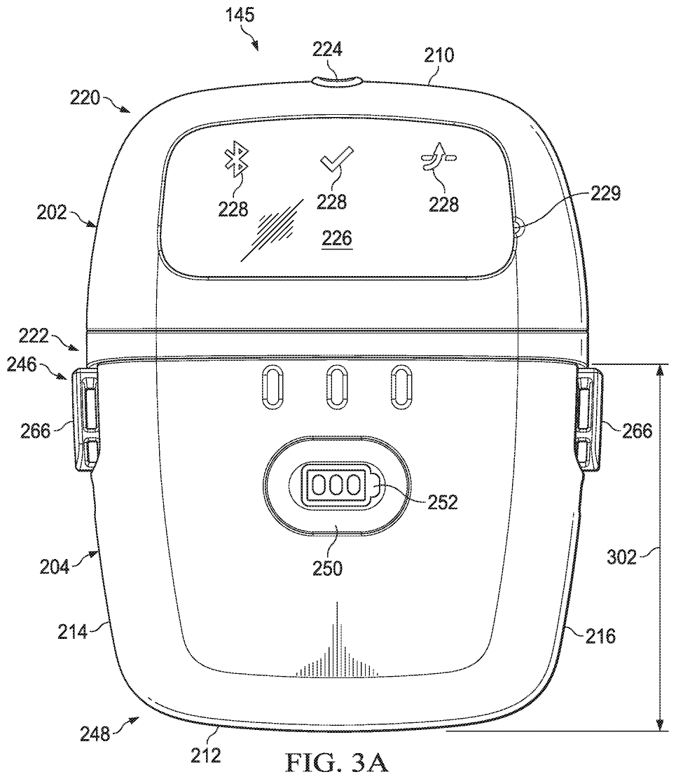

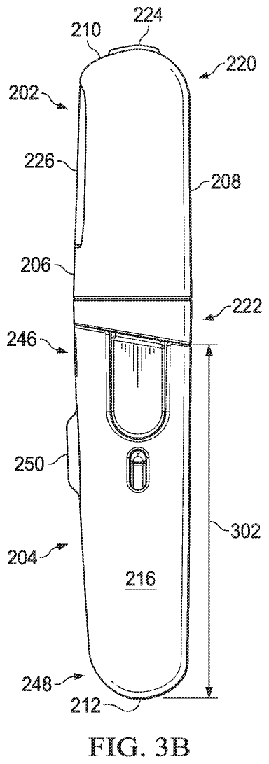

Referring to FIGS. 3A and 3B, another embodiment of the therapy device 145 is shown. The therapy device 145 may include the pump housing 202 and the power module 204. The power module 204 may have a length 302 that may be defined between the first end 246 and the second end 248 of the power module 204. The length 302 may be less than the length 249 of the therapy device 145 of FIGS. 2A-2F. The length 302 of the power module 204 of FIGS. 3A and 3B may be less than the length 249 of the power module 204 of FIGS. 2A-2F because a battery of the power module 204 of FIGS. 3A and 3B may be smaller than the battery 254 of the power module 204 of FIGS. 2A-2F. The therapy device 145 of FIGS. 3A and 3B may have a lower battery capacity than the therapy device 145 of FIGS. 2A-2F. In some embodiments, the power module 204 may be adapted to house batteries of different capacities such that the therapy device 145 may be larger when a higher capacity is desired and may be smaller when a lower capacity is desired. In some instances, it may be desirable to have the therapy device 145 with a smaller capacity because it may be easier for a user to conceal or to carry. In other instances, it may be desirable for the therapy device 145 to have a larger capacity because the therapy device 145 may need to be re-charged less often and the user may be able to go for longer periods of time without worrying about the battery life of the therapy device 145.

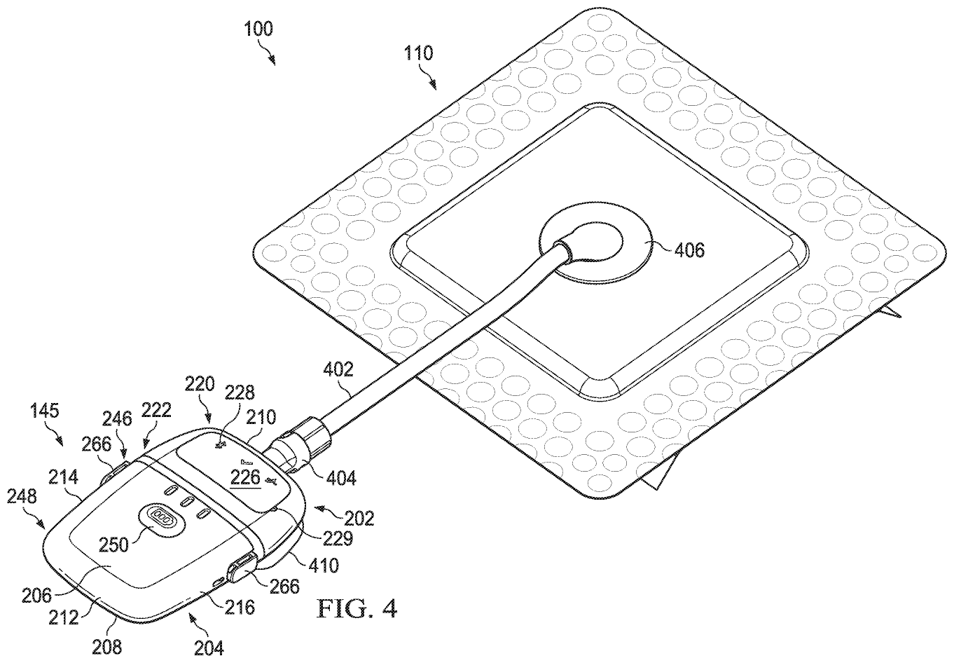

Referring to FIG. 4, the therapy system 100 is shown. The therapy system 100 may include the dressing 110 coupled to the therapy device 145. The therapy device 145 may be coupled to the dressing 110 with a conduit 402. In some embodiments, the conduit 402 may be coupled to a therapy device connection 404 that may couple with the dressing connection 224 of the pump housing 202. Similarly, the conduit 402 may be coupled to a dressing interface 406 opposite the therapy device connection 404. In some embodiments, the pump 230 may be actuated to deliver negative pressure to the dressing 110 through the conduit 402.

In some embodiments, the therapy device 145 may include a base 410. The base 410 may be coupled to the second surface 208 of the therapy device 145. The therapy device 145 may sit on the base 410 such that the base 410 provides stability for the therapy device 145.

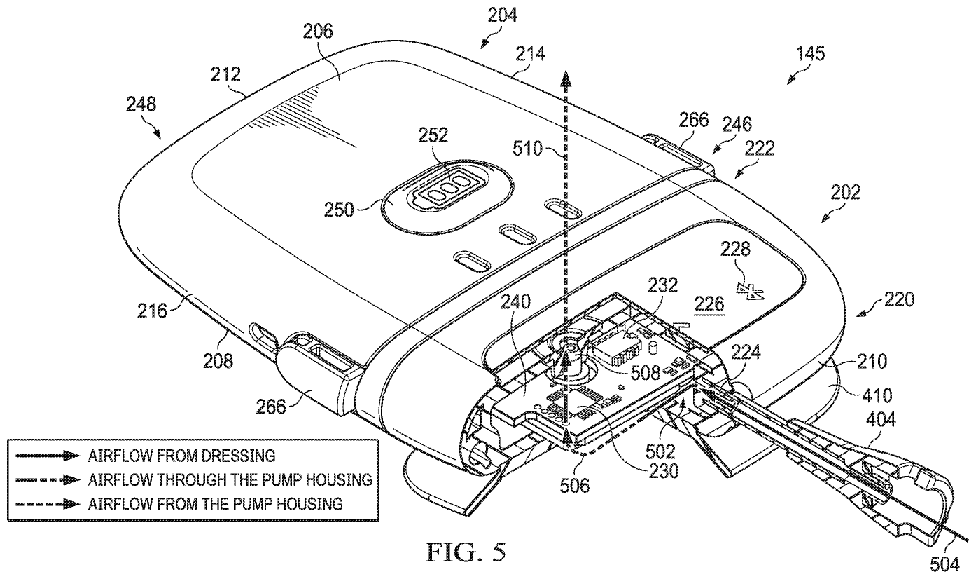

Referring to FIG. 5, the therapy device 145 is shown with the pump housing 202 including an optional sealed architecture. For example, the pump housing 202 may include an interior chamber 502 that may be hermetically sealed from an ambient environment. An inlet of the pump 230 may be directly exposed to the interior chamber 502 to generate negative pressure within the interior chamber 502 and an airflow 504 from the dressing 110 when the pump 230 is actuated. The airflow 504 may be drawn into the interior chamber 502 of the pump housing 202 and transition to an interior airflow 506 within the interior chamber 502. The interior airflow 506 may flow freely throughout the interior chamber 502 of the pump housing 202. The interior chamber 502 may include a one-way valve such as valve 508 that may enable the exhaust from an outlet of the pump 230 to be released from the interior chamber 502 of the pump housing 202. The valve 508 may couple to the port 229 such that the interior airflow 506 may travel from the inlet to the outlet of the pump 230 and through the valve 508 and escape from the pump housing 202 through the port 229. Once the interior airflow 506 has traveled through the port 229, it may become an exhaust airflow 510 that may combine with the ambient environment surrounding the therapy device 145.

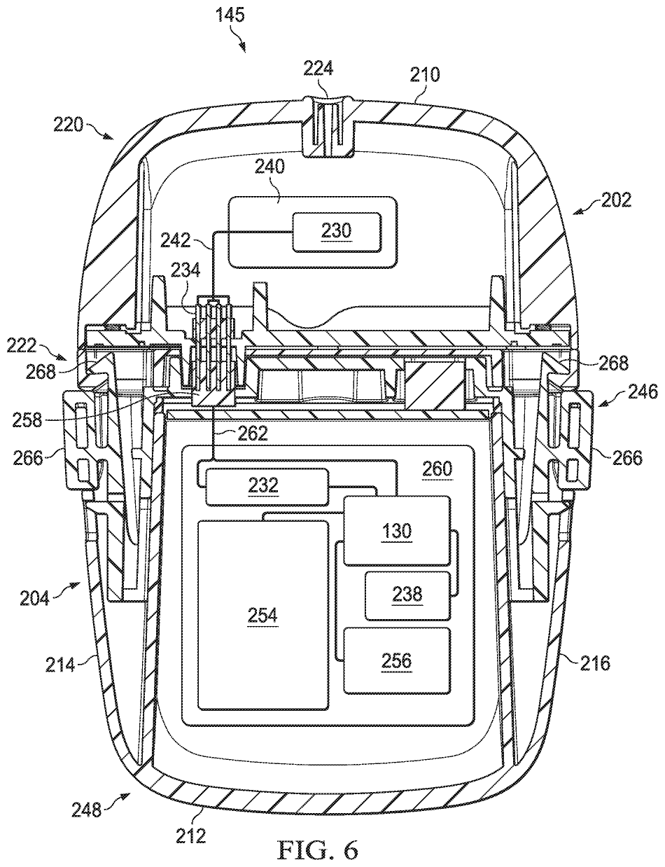

Referring to FIG. 6, another embodiment of the therapy device 145 is shown. The exterior structure of the therapy device 145 may be substantially similar to that described above with reference to FIGS. 2A-2F. For example, the therapy device 145 may include the pump housing 202 and the power module 204. In some embodiments, the internal components of the therapy device 145 may be disposed in different locations. For example, the pump housing 202 may include the pump 230 and the first communications connector 234 and the power module 204 may include the controller 130, the first pressure sensor 232, the battery 254, the second pressure sensor 256, and the second communications connector 258. In some embodiments, the power module 204 may further include the wireless communication device 238. In embodiments where the pump housing 202 only includes the pump 230 and the first communications connector 234, the pump housing 202 may be considered disposable and may be discarded if any fluid or other contaminants from the dressing 110 flow into the pump housing 202 while the pump 230 is actuated.

The pump 230 may be coupled to the printed circuit board 240. Additionally, the pump 230 may be communicatively coupled with the first communications connector 234. In some embodiments, the wire 242 may couple the pump 230 to the first communications connector 234. In other embodiments, the pump 230 may be wirelessly coupled with the first communications connector 234. The controller 130, the first pressure sensor 232, the battery 254, the second pressure sensor 256, and the wireless communication device 238 may each be coupled to the printed circuit board 260. The controller 130 may be configured to send signals to and receive signals from each of the first pressure sensor 232, the battery 254, the second pressure sensor 256, and the wireless communication device 238. Further, the controller 130 may be able to send signals to and receive signals from the pump 230 via the second communications connector 258 and the first communications connector 234. In some embodiments, the first pressure sensor 232 may be configured to sense the pressure at the pump 230 through the second communications connector 258 and the first communications connector 234. In other embodiments, there may be a fluid path separate from the second communications connector 258 and the first communications connector 234 that couples the first pressure sensor 232 to the pump 230 so that the first pressure sensor 232 is able to sense a pressure at the pump 230.

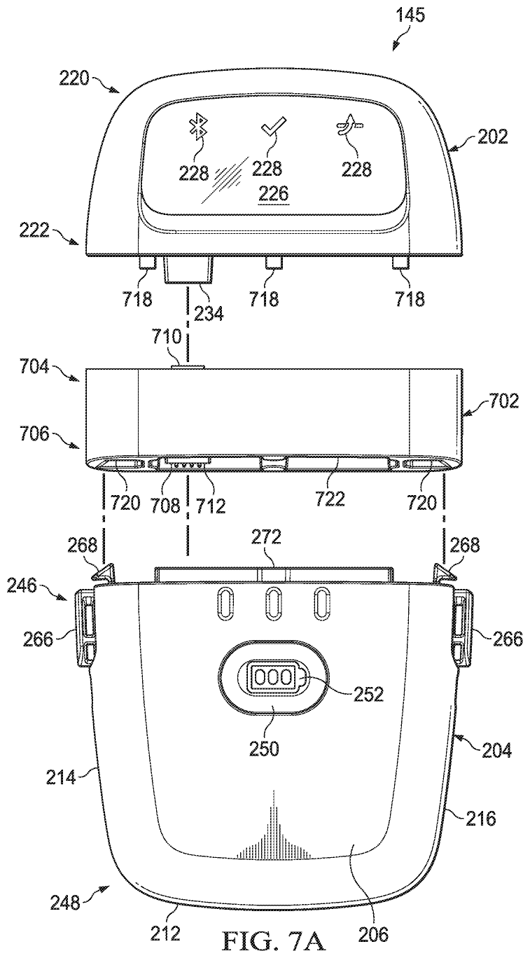

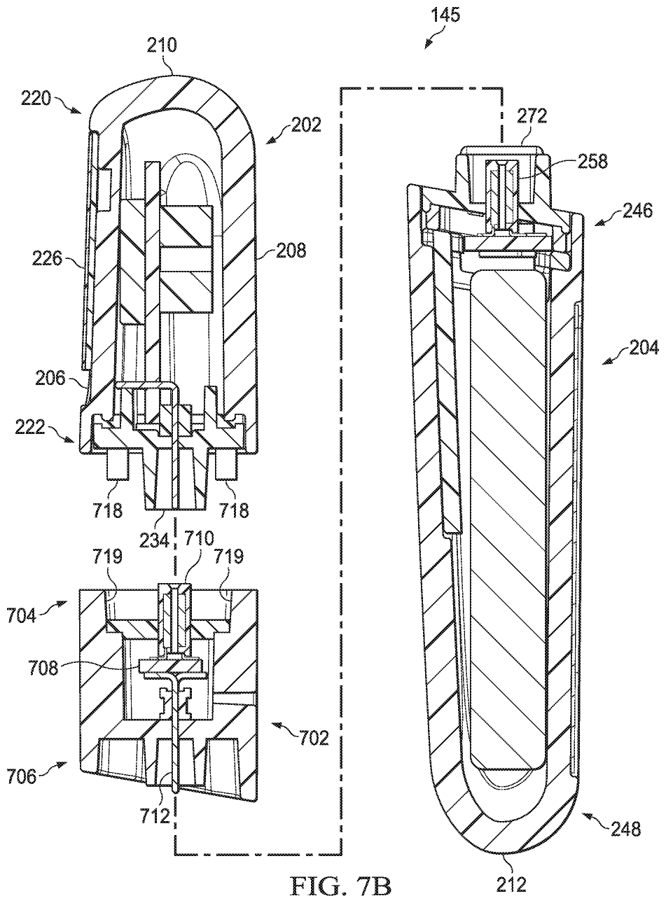

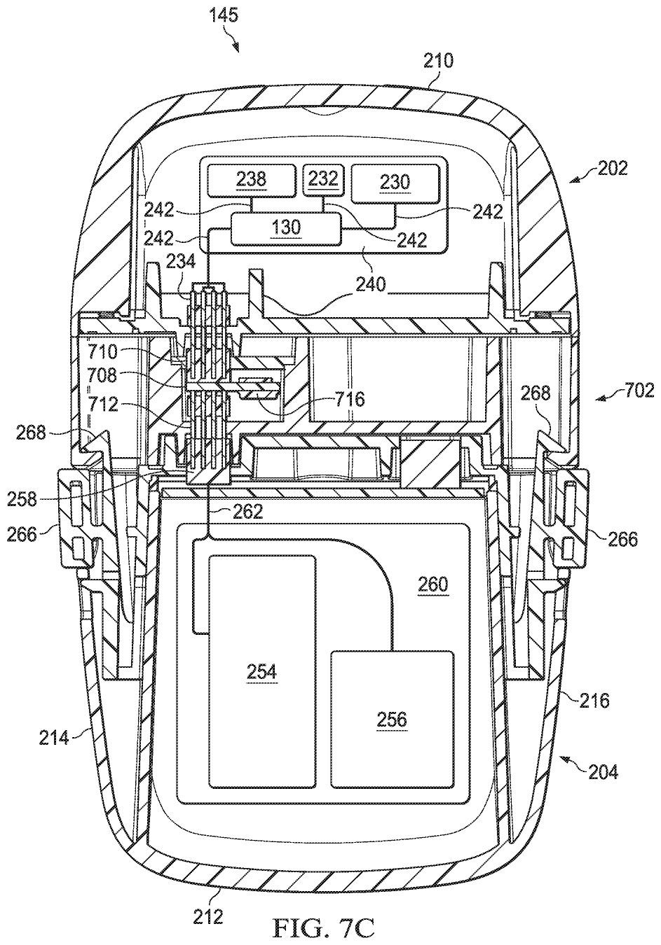

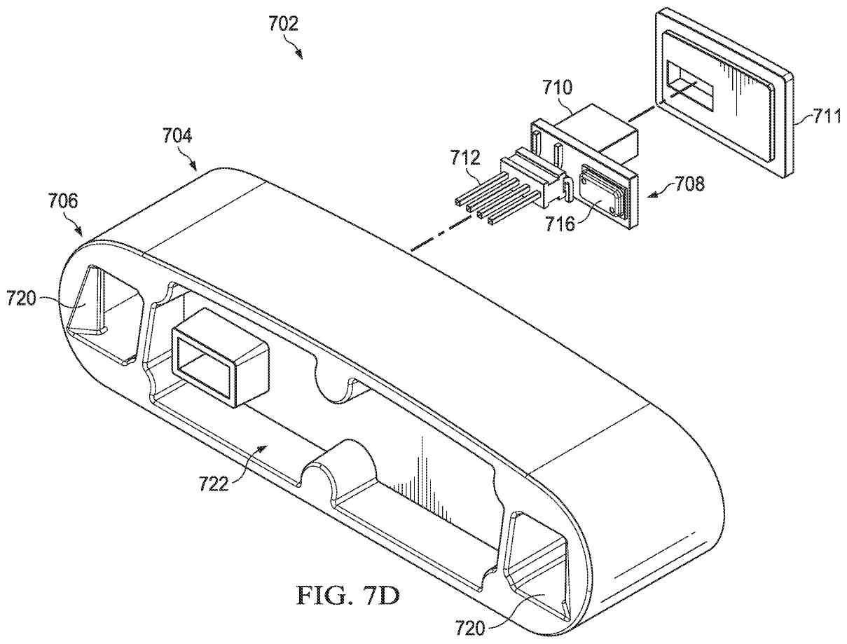

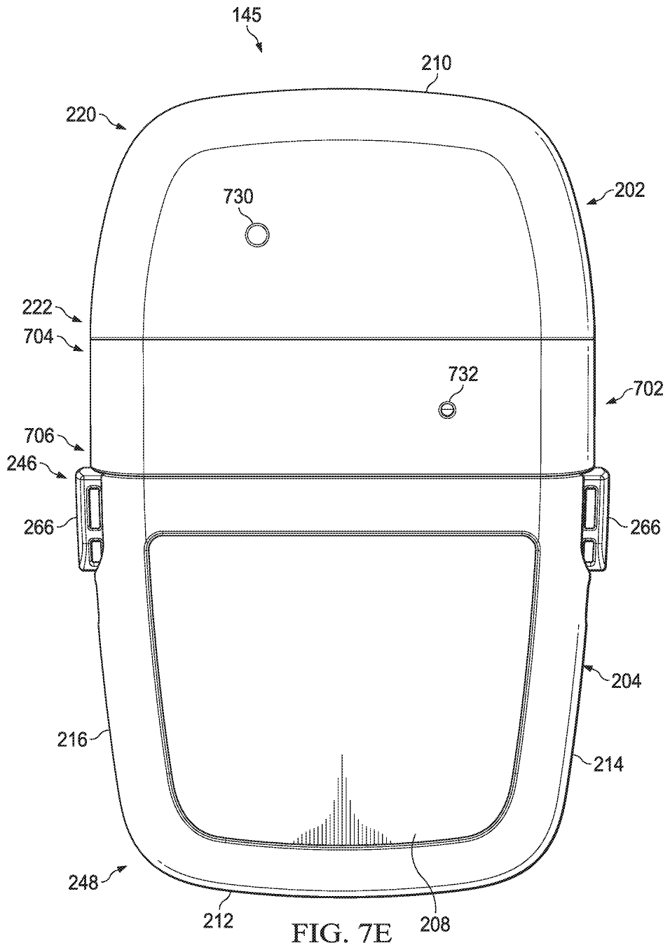

Referring to FIGS. 7A-7D, another embodiment of the therapy device 145 is shown. The therapy device 145 may include the pump housing 202, the power module 204 and a pressure sensing module 702. The pressure sensing module 702 may be configured to be coupled between the pump housing 202 and the power module 204. More specifically, the pressure sensing module 702 may have a first end 704 that may couple to the second end 222 of the pump housing 202 and a second end 706 that may couple to the first end 246 of the power module 204.

The pressure sensing module 702 may include a third communications connector 708 that may extend from the first end 704 of the pressure sensing module 702 to the second end 706 of the pressure sensing module 702. In some embodiments, the third communications connector 708 may include a first end 710 that may couple to the first communications connector 234 through a sealing element or a sealing lid 711 and a second end 712 that may couple to the second communications connector 258. In some embodiments, the pressure sensing module 702 may further include a third pressure sensor 716. The third pressure sensor 716 may be communicatively coupled to the third communications connector 708 such that the third pressure sensor 716 can be communicatively coupled with the controller 130. In some embodiments, the pressure sensing module 702 may further include a second pump (not shown). The second pump may be analogous to the pump 230 and may work in conjunction with the pump 230 to deliver negative pressure from the therapy device 145 to the dressing 110. In some embodiments, the second pump may be included where the therapy device 145 is configured to work with wounds or tissue sites that require a higher flow to be generated by the therapy device 145.

In some embodiments, the pressure sensing module 702 may be coupled to the pump housing 202 via one or more projections 718 that extend from the second end 222 of the pump housing 202 towards the pressure sensing module 702. The one or more projections 718 may be received by one or more corresponding recesses 719 in the pressure sensing module 702. Further, the pump housing 202 may not include the center recess 274 and the first communications connector 234 may extend past the second end 222 of the pump housing 202 to couple with the first end 710 of the third communications connector 708.

In some embodiments, the pressure sensing module 702 may be releasably coupled to the power module 204 with the snaps 266. In some embodiments, the snaps 266 may be part of the power module 204 and may be depressed into the power module 204 to release the pressure sensing module 702 from the power module 204. When the snaps 266 are depressed into the power module 204, the hooks 268 may be released from recesses 720 of the pressure sensing module 702. In other embodiments, the snaps 266 may be manipulated in another way to release the power module 204 from the pressure sensing module 702. In other embodiments, not pictured herein, the snaps 266 may be disposed within the pressure sensing module 702 or the power module 204 such that a user cannot access the snaps 266. When disposed within the pressure sensing module 702 or the power module 204 the snaps 266 may couple the pressure sensing module 702 to the power module 204 but the pressure sensing module 702 and the power module 204 may not be separated by a user. In some embodiments, the pressure sensing module 702 may further include a central recess 722 that may be configured to receive the extension 272 of the power module 204. The second end 712 of the third communications connector 708 may be disposed within the central recess 722 and may couple to the second communications connector 258 when the power module 204 is coupled to the pressure sensing module 702.

In some embodiments, the therapy device 145 may have a first dressing connector 730 and a second dressing connector 732 disposed on the second surface 208 of the therapy device 145. The first dressing connector 730 may be disposed through the pump housing 202 and the second dressing connector 732 may be disposed through the pressure sensing module 702. In some embodiments, the first dressing connector 730 may fluidly couple the pump housing 202 to the dressing 110 and the second dressing connector 732 may fluidly couple the pressure sensing module 702 to the dressing 110. The first dressing connector 730 and the second dressing connector 732 may be coupled to a conduit or to another device that may enable the pump housing 202 and the pressure sensing module 702 to be fluidly coupled to the dressing 110.

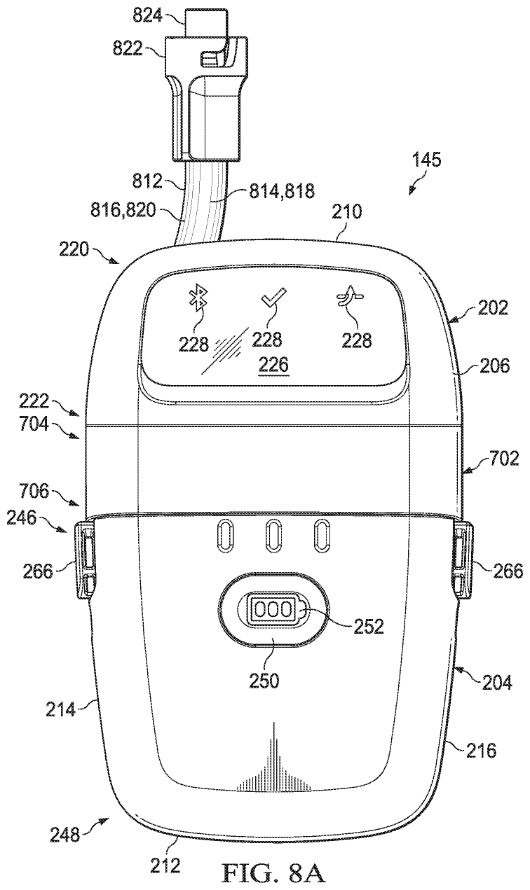

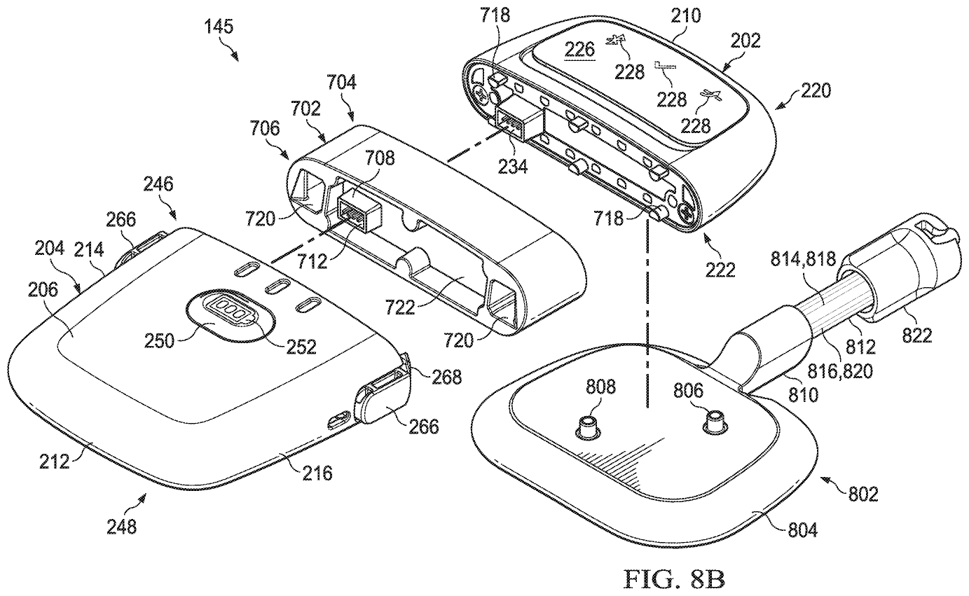

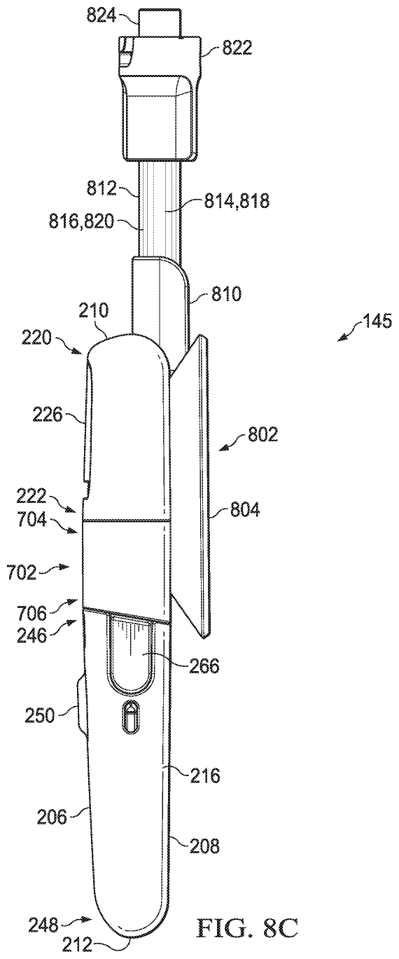

Referring to FIGS. 8A and 8B, the therapy device 145 of FIGS. 7A-7E is shown coupled to a dressing connector 802. The dressing connector 802 may include a base 804 that may include a first connector 806 and a second connector 808. The first connector 806 may be configured to couple with the first dressing connector 730 of the therapy device 145 and the second connector 808 may be configured to couple with the second dressing connector 732 of the therapy device 145. The first connector 806 may couple to the first dressing connector 730 to couple the first connector 806 in fluid communication with the pump 230 within the pump housing 202. The second connector 808 may couple to the second dressing connector 732 to couple to the second connector 808 in fluid communication with the third pressure sensor 716 within the pressure sensing module 702.

The dressing connector 802 may further include a conduit connection 810 that may couple to a conduit 812. The conduit 812 may be a multi-lumen conduit in some embodiments. For example, the conduit 812 may include a central lumen 814 that may be surrounded by one or more surrounding lumens 816. In some embodiments, the central lumen 814 may provide a negative pressure pathway 818 that may extend from the pump 230 through the first dressing connector 730 and the first connector 806 into the central lumen 814 and to the dressing 110. The negative pressure pathway 818 may be configured to fluidly couple the first connector 806 and the pump 230 to the dressing 110. In embodiments where the pressure sensing module 702 includes the second pump (not shown), the second pump may also be in fluid communication with the negative pressure pathway 818. The surrounding lumens 816 may provide a sensing pathway 820 that may extend from the third pressure sensor 716 through the second dressing connector 732 and the second connector 808 into the surrounding lumens 816 and to the dressing 110. The sensing pathway 820 may be configured to fluidly couple the second connector 808 and the third pressure sensor 716 to the dressing 110. In some embodiments, the central lumen 814 may be fluidly isolated from the surrounding lumens 816 such that the negative pressure pathway 818 is fluidly isolated from the sensing pathway 820.

In some embodiments, the sensing pathway 820, the third pressure sensor 716, the first pressure sensor 232, and the controller 130 may define a blockage detection module. The sensing pathway 820 may allow the third pressure sensor 716 to sense a pressure at the dressing 110. The third pressure sensor 716 may communicate the pressure at the dressing 110 with the controller 130 of the therapy device 145. In some embodiments, the controller 130 may be configured to compare the pressure at the dressing 110 to a pressure sensed by the first pressure sensor 232 at the pump 230. If the pressure sensed by the first pressure sensor 232 is different from the pressure sensed by the third pressure sensor 716 by a predetermined value, the therapy device 145 may output an indication to a user that the therapy system 100 needs to be checked. For example, if the pressure sensed by the third pressure sensor 716 is different from the pressure sensed by the first pressure sensor 232 by a predetermined value, there may be a blockage within the dressing 110 or between the therapy device 145 and the dressing 110. The user may be alerted that a blockage has been detected and the user may be able to clear the blockage so the therapy system 100 operates as intended.

In some embodiments, the dressing connector 802 may further include an extender 822. The extender 822 may be configured to couple the conduit 812 to a second conduit 824 that may couple to the dressing 110. In other embodiments, the extender 822 may not be included and the conduit 812 may be directly coupled to the dressing 110.

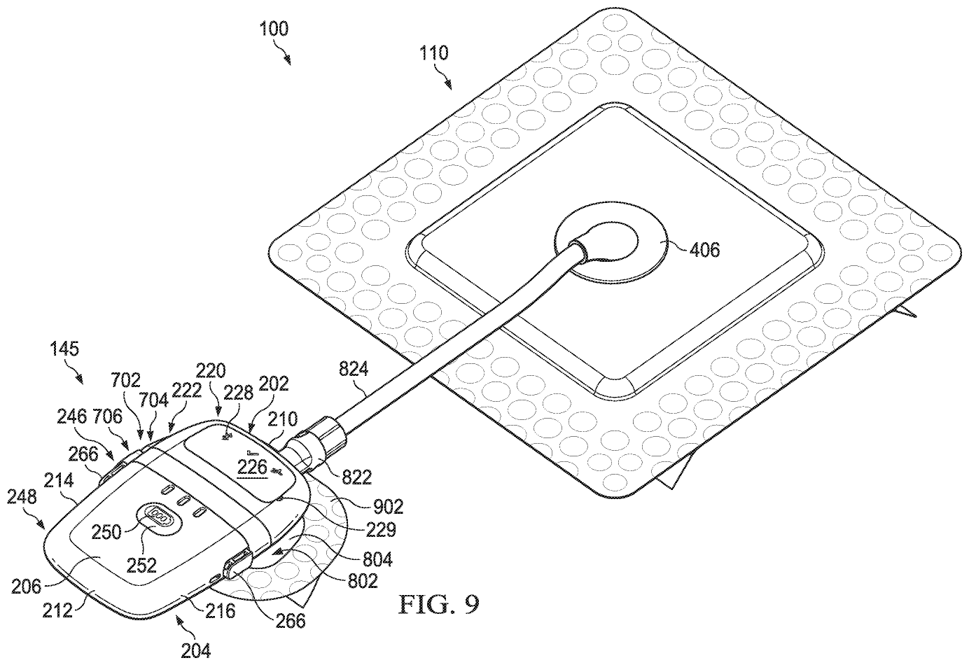

Referring to FIG. 9, the therapy system 100 is shown with the therapy device 145 of FIGS. 7A-7E coupled to the dressing 110. The dressing 110 may be an absorbent dressing that may be adapted to store fluid from a tissue site within the dressing 110.

The therapy device 145 may be coupled to the dressing connector 802 such that the negative pressure pathway 818 is fluidly coupling the pump 230 to the dressing 110 and the sensing pathway 820 is fluidly coupling the third pressure sensor 716 to the dressing 110. In some embodiments, the dressing connector 802 may be coupled to a film 902. The film 902 may be adapted to couple to a patient or it may be adapted to couple to another surface such that the therapy device 145 is stationary during treatment of a tissue site. In some embodiments, the film 902 may include an attachment device on a surface opposite the dressing connector 802. The attachment device may be a medically-acceptable, pressure-sensitive adhesive configured to bond the film 902 to the patient. In some embodiments, for example, some or all of the film 902 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

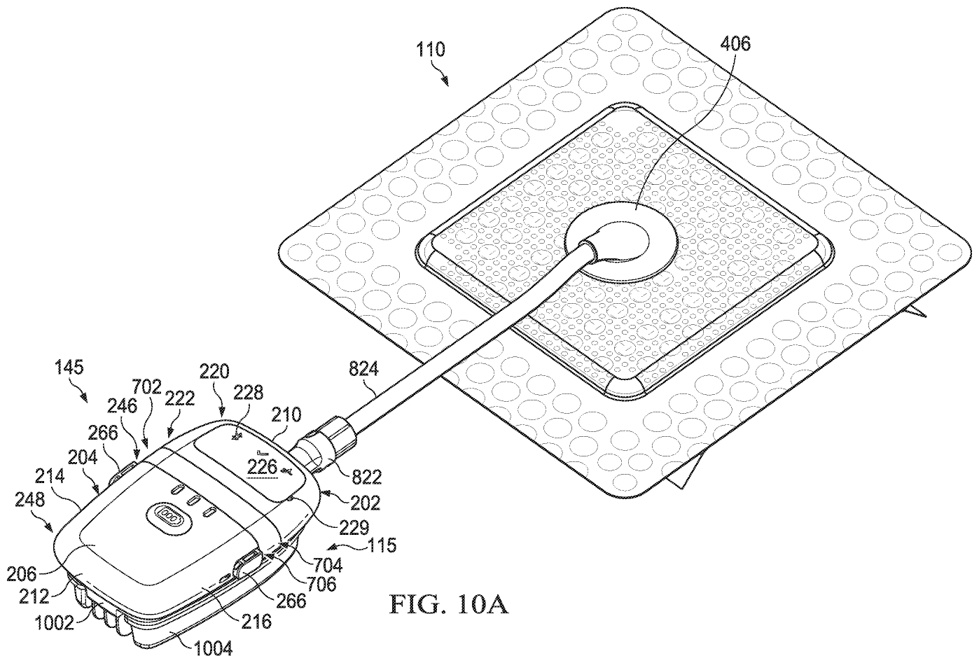

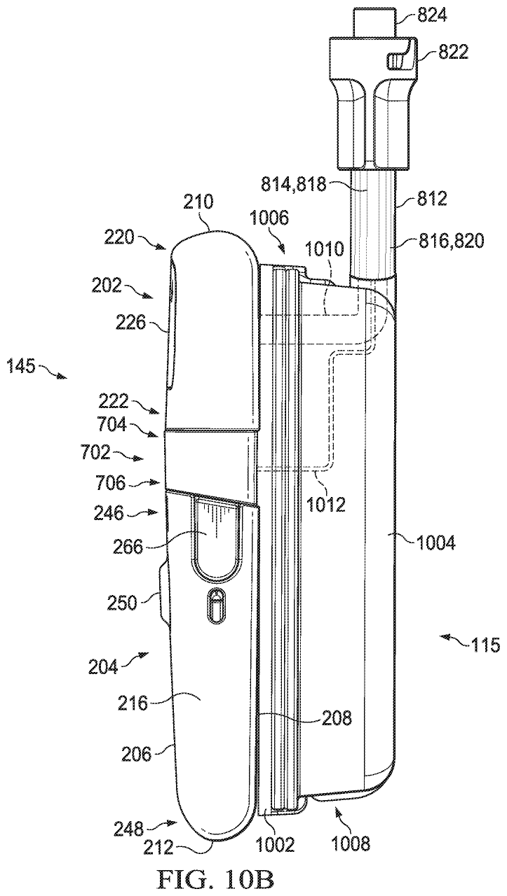

Referring to FIGS. 10A-10C, another embodiment of the therapy system 100 is shown. The therapy device 145 may be coupled to the dressing 110 with the conduit 812, the extender 822, and the second conduit 824. The dressing 110 may not be an absorbent dressing and fluid from the tissue site may be configured to flow through the dressing 110, the second conduit 824, and the conduit 812 to be contained and stored in a canister or container such as the container 115. In some embodiments, the container 115 may include a lid 1002 and a body 1004. The lid 1002 may be coupled to the second surface 208 of the therapy device 145. The container 115 may have a first end 1006 and a second end 1008 opposite the first end 1006. The first end 1006 of the container 115 may be configured to couple with the conduit 812. As described above, the conduit 812 may be a multi-lumen conduit in some embodiments. For example, the conduit 812 may include the central lumen 814 that may be surrounded by the surrounding lumens 816. The conduit 812 may be coupled to the extender 822 in some embodiments and the second conduit 824 may be coupled between the extender 822 and the dressing interface 406.

In some embodiments, a negative pressure lumen 1010 may extend from the first dressing connector 730 through the container 115 to the conduit 812. The negative pressure lumen 1010 may be included in the negative pressure pathway 818 such that the pump 230 is fluidly coupled to the dressing 110 through the negative pressure lumen 1010. A pressure sensing lumen 1012 may also be included in some embodiments. The pressure sensing lumen 1012 may extend from the second dressing connector 732 through the container 115 to the conduit 812. The pressure sensing lumen 1012 may be included in the sensing pathway 820 such that the third pressure sensor 716 is fluidly coupled to the dressing 110 through the pressure sensing lumen 1012. The pressure sensing lumen 1012 may be configured such that an interior of the pressure sensing lumen 1012 is fluidly isolated from an interior of the container 115 and from the negative pressure lumen 1010.

Also described herein is a method of treating a tissue site with negative pressure. In some example embodiments, the method may include obtaining the pump housing 202 that may include the pump 230 configured to generate negative pressure, the first pressure sensor 232 configured to sense a pressure at the pump 230, and the controller 130 operably coupled to the pump 230 and the first pressure sensor 232 within an interior of the pump housing 202. The method may further include obtaining the power module 204 including the battery 254 configured to provide power to the therapy device 145 and the second pressure sensor 256 configured to sense an ambient pressure. The method may further include coupling the pump housing 202 to the power module 204 to form the therapy device 145. The battery 254 and the second pressure sensor 256 may be operably connected to the controller 130 in some embodiments. The method may further include fluidly coupling the pump 230 to the dressing 110 disposed at the tissue site, actuating the pump 230 to deliver negative pressure to the dressing 110, and monitoring, with the therapy device 145, the dressing 110 while the pump 230 is delivering negative pressure to the dressing 110.

In some example embodiments, the method may further include coupling the pressure sensing module 702 between the pump housing 202 and the power module 204 of the therapy device 145. The pressure sensing module 702 may include the third pressure sensor 716. In some example embodiments, coupling the therapy device 145 to the dressing 110 disposed at the tissue site may include coupling the therapy device 145 to the dressing connector 802. The dressing connector 802 may include the first connector 806 and the second connector 808. The dressing connector 802 may be coupled to the conduit 812 that may include the negative pressure pathway 818 and the sensing pathway 820. The first connector 806 can be configured to couple to the pump housing 202 such that the first connector 806 is in fluid communication with the pump 230. The second connector 808 may be configured to couple to the pressure sensing module 702 such that the second connector 808 is in fluid communication with the third pressure sensor 716. The negative pressure pathway 818 may be configured to fluidly couple the first connector 806 and the pump 230 to the dressing 110. The sensing pathway 820 may be configured to fluidly couple the second connector 808 and the third pressure sensor 716 to the dressing 110. In some example embodiments, the method may further include collecting fluid from the tissue site in the container 115 of the therapy device 145. The container 115 may be configured to be coupled in fluid communication with the negative pressure pathway 818 between the pump 230 and the dressing 110. In some example embodiments, monitoring with the therapy device 145, the dressing 110 while the pump 230 is delivering negative pressure to the dressing 110 may include monitoring the pressure at the tissue site with the pressure sensing module 702.

In some example embodiments, monitoring, with the therapy device 145, the dressing 110 while the pump 230 is delivering negative pressure to the dressing 110 may include using a blockage detection module of the therapy device 145 to detect blockages within the dressing 110 or between the therapy device 145 and the dressing 110.

The systems, apparatuses, and methods described herein may provide significant advantages. For example, the therapy device 145 may provide a modular device that can be modified to use with different types of dressings and wounds. The therapy device 145 can be modified to provide greater or less battery capacity which may result in the therapy device 145 being sized differently depending on the battery capacity. The therapy device 145 may be supplied in discrete parts or may be supplied as an assembled unit. The therapy device 145 can be modified as health care providers see fit and provides a solution that is in one product for ease of use and flexibility.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A therapy device comprising:
a pump housing comprising:
a pump configured to deliver negative pressure to a dressing,
a first pressure sensor configured to sense a pressure at the pump,
a controller operably coupled to the pump and the first pressure sensor within an interior of the pump housing, and
a first communications connector configured to provide communication with the controller from an exterior of the pump housing; and
a power module configured to couple to the pump housing, the power module comprising:
a battery configured to provide power,
a second pressure sensor configured to sense an ambient pressure, and
a second communications connector configured to be coupled to the first communications connector to operably connect the battery and the second pressure sensor to the controller.

2. (canceled)

3. (canceled)

4. The therapy device of claim 1, wherein the pump housing further comprises a wireless communication device operably coupled to the controller.

5. (canceled)

6. The therapy device of claim 1, wherein the pump housing further comprises a dressing connection configured to fluidly couple the pump to the dressing.

7. The therapy device of claim 1, further comprising a pressure sensing module, the pressure sensing module configured to be coupled between the pump housing and the power module.

8. The therapy device of claim 7, further comprising a fluid storage container configured to be fluidly coupled between the pump and the dressing.

9. The therapy device of claim 7, wherein the pressure sensing module comprises a third pressure sensor and a third communications connector, and wherein the third communications connector is configured to be coupled between the first communications connector and the second communications connector to operably connect the third pressure sensor to the controller.

10. The therapy device of claim 9, further comprising a dressing connector configured to couple the therapy device to the dressing, the dressing connector comprising:
a first connector configured to couple to the pump housing in fluid communication with the pump;
a second connector configured to couple to the pressure sensing module in fluid communication with the third pressure sensor;
a negative pressure pathway configured to fluidly couple the first connector and the pump to the dressing; and
a sensing pathway configured to fluidly couple the second connector and the third pressure sensor to the dressing.

11. The therapy device of claim 10, wherein at least a portion of the negative pressure pathway and the sensing pathway are carried by a multi-lumen conduit.

12. The therapy device of claim 10, wherein the sensing pathway is fluidly isolated from the negative pressure pathway.

13. The therapy device of claim 10, further comprising a fluid storage container configured to be coupled in fluid communication with the negative pressure pathway between the pump and the dressing.

14. The therapy device of claim 10, further comprising a second pump in fluid communication with the negative pressure pathway, wherein the second pump is positioned within the pressure sensing module.

15. The therapy device of claim 1, further comprising a blockage detection module configured to detect blockages within the dressing or between the therapy device and the dressing.

16. The therapy device of claim 1, wherein the pump housing has an interior chamber that is hermetically sealed from ambient environment.

17. A system for treating a tissue site with negative pressure, the system comprising:
a dressing configured to couple to the tissue site;
a pump housing comprising:
a pump configured to deliver negative pressure to the dressing, and
a first communications connector configured to provide communication with the pump from an exterior of the pump housing; and
a power module configured to couple to the pump housing, the power module comprising:
a battery configured to provide power,
a first pressure sensor configured to sense a pressure at the pump,
a second pressure sensor configured to sense an ambient pressure,
a controller operably coupled to the battery, the first pressure sensor, and the second pressure sensor within an interior of the power module, and
a second communications connector configured to be coupled to the first communications connection to operably connect the controller and the first pressure sensor to the pump.

18. A method of treating a tissue site with negative pressure, the method comprising:
obtaining a pump housing, comprising:
a pump configured to generate negative pressure,
a first pressure sensor configured to sense a pressure at the pump, and
a controller operably coupled to the pump and the first pressure sensor within an interior of the pump housing;
obtaining a power module, comprising:
a battery configured to provide power, and
a second pressure sensor configured to sense an ambient pressure;
coupling the pump housing to the power module to form a therapy device, wherein the battery and the second pressure sensor are operably connected to the controller;
fluidly coupling the pump to a dressing disposed at the tissue site;
actuating the pump to deliver negative pressure to the dressing; and
monitoring, with the therapy device, the dressing while the pump is delivering negative pressure to the dressing.

19. The method of claim 18, further comprising coupling a pressure sensing module between the pump housing and the power module of the therapy device, the pressure sensing module comprising a third pressure sensor.

20. The method of claim 19, wherein coupling the therapy device to the dressing disposed at the tissue site comprises coupling the therapy device to a dressing connector, the dressing connector comprising:
a first connector configured to couple to the pump housing in fluid communication with the pump;
a second connector configured to couple to the pressure sensing module in fluid communication with the third pressure sensor;
a negative pressure pathway configured to fluidly couple the first connector and the pump to the dressing; and
a sensing pathway configured to fluidly couple the second connector and the third pressure sensor to the dressing.

21. The method of claim 20, further comprising collecting fluid from the tissue site in a canister of the therapy device, the canister configured to be coupled in fluid communication with the negative pressure pathway between the pump and the dressing.

22. The method of claim 19, wherein monitoring, with the therapy device, the dressing while the pump is delivering negative pressure to the dressing comprises monitoring the pressure at the tissue site with the pressure sensing module.

23. The method of claim 18, wherein monitoring, with the therapy device, the dressing while the pump is delivering negative pressure to the dressing comprises using a blockage detection module of the therapy device to detect blockages within the dressing or between the therapy device and the dressing.

24. (canceled)
